(19) United States
(12) Patent Application Publication
(10) Pub. No.: US 2004/0138204 A1
(43) Pub. Date: Jul. 15, 2004

(54) Compositions and methods for pain reduction

(76) Inventor: James Frederick Harrington JR., Providence, RI (US)

Correspondence Address: Ingrid A. Beattie, Ph.D., J.D., Mintz, Levin, Cohn, Ferris,, Glovsky and Popeo, P.C., One Financial Center, Boston, MA, 02111, US

(21) Appl. No.: 10/695,680

(22) Filed: Oct. 29, 2003

Related U.S. Application Data
(60) Provisional application No. 60/422,224, filed on Oct. 30, 2002.

(51) Int. Cl. A61K 31/55; A61K 31/425

(52) U.S. Cl. 514/217.03

(57) ABSTRACT

The invention provides compositions and methods for inhibiting the binding of free glutamate to a glutamate receptor on a neuronal cell by contacting a neuronal tissue with a glutamate receptor antagonist.

### RELATED APPLICATIONS

This application claims priority to provisional patent application serial No. 60/422,224, filed on Oct. 30, 2002, the entire contents of which are hereby incorporated by reference.

### BACKGROUND OF THE INVENTION

The invention relates to pain management.

The current gold standard for treatment of sciatic pain is surgical removal of the herniated disc fragment from the environs of the nerve root in the epidural space. Though often effective, the operation has risks of nerve injury and mechanical disruption of low back function leading to mechanical back pain. It also is expensive. It is estimated that over 100,000 such operations are performed each year in the United States.

### SUMMARY OF THE INVENTION

The invention is based on the discovery that sciatic pain from lumbar disc herniations was related to more than simple nerve pressure. A chemical component, free glutamate liberated from degenerating cartilage, was found to be involved in lumbar radiculopathy and in other aspects of mechanical low back pain.

Accordingly, the invention provides compositions and methods for inhibiting the binding of free glutamate to a glutamate receptor by contacting a dorsal root ganglion cell or other spine-associated neuronal tissue or cell with an ionotropic glutamate receptor antagonist. For example, the ionotropic glutamate receptor antagonist is a non-N-methyl-D-aspartate (NMDA) type receptor antagonist such as a alpha-amino-3-hydroxy-5-methyl-4-isoxalone propionate (AMPA) receptor antagonist or a kainate-activated (KA) receptor antagonist. Alternatively, the antagonist is a metabotropic glutamate receptor antagonist. In various embodiments, the composition does not contain an NMDA type receptor antagonist. The composition preferentially inhibits glutamate binding to a metabotropic glutamate receptor compared to an ionotropic glutamate receptor. Alternatively, composition preferentially inhibits glutamate binding to a ionotropic glutamate receptor compared to an metabotropic glutamate receptor. For example, the inhibitor preferentially reduces metabotropic glutamate receptor binding by at least 10%, more preferably 20%, 50%, 100%, and 200% compared to the level of reduction of ionotropic glutamate receptor binding. In another example, the inhibitor preferentially reduces ionotropic glutamate receptor binding by at least 10%, more preferably 20%, 50%, 100%, and 200% compared to the level of reduction of metabotropic glutamate receptor binding. Preferably, the compositions preferentially inhibit binding to a target receptor subtype. The compositions are suitable for administration, e.g., injection, into joint tissue or intervetebral disc tissue.

The compositions and methods are used to alleviate pain in a mammal, e.g., a human subject that is suffering from or at risk of developing back pain, joint pain, or sciatic pain. Perception of pain in a human subject is identified and evaluated using known methods, e.g., a visual analog pain scale and/or the SF-36 health questionnaire. An improvement in the pain index indicates that pain is alleviated. For example, the pain is associated with a herniated disc. A herniated disc is a displaced fragment of nucleus pushed out through a tear in the outer layer of the disc (annulus). For a disc to become herniated, it typically is in an early stage of degeneration. The pain one feels down the leg is termed sciatica or sciatic pain.

Antagonists are administered as pain relievers for sciatic pain and non-sciatic pain, e.g., in the latter case, by contacting glutamate receptors located in the disc annulus. The antagonist is administered into an epidural space. Alternatively, the antagonist is administered into the spinal fluid rather than into an epidural space.

A glutamate receptor antagonist is a compound that inhibits binding of glutamate with a cell-bound glutatmate receptor. For example, a glutamate receptor interacts with a free glutamate or a cellular glutamate receptor (or subunit thereof) on the surface of a neuronal cell and reduces the ability of the natural ligand to stimule a response pathway within the cell, e.g. by interfering with the binding of L-glutamate to a cell-bound receptor.

The antagonist is an organic polypeptide, e.g., a molecule or a fragment of a glutamate receptor or subunit thereof. The compounds described herein are substantially pure. By a substantially pure polypeptide is meant a polypeptide, which is separated from those components (proteins and other naturally-occurring organic molecules), which naturally accompany it. A polypeptide is substantially pure when it constitutes at least 60%, by weight, of the protein in the preparation. Preferably, the protein in the preparation is at least 75%, more preferably at least 90%, and most preferably at least 99%, by weight, of the desired peptide. A substantially pure polypeptide is obtained, e.g., by extraction from a natural source; by expression of a recombinant nucleic acid; or by chemically synthesizing the protein. Purity is measured by a number appropriate methods known in the art, e.g., column chromatography, polyacrylamide gel electrophoresis, or HPLC analysis. A protein is substantially free of naturally associated components when it is separated from those contaminants, which accompany it in its natural state. Thus, a protein which is chemically synthesized or produced in a cellular system different from the cell from which it naturally originates is substantially free from its naturally associated components.

In addition to peptides, the invention encompasses nucleic acids, e.g., oligonucleotides, which encode glutamate receptor antagonists. The nucleic acids, e.g., DNA or RNA, are substantially pure. By substantially pure DNA is meant DNA that is free of the genes, which, in the naturally-occurring genome of the organism from which the DNA of the invention is derived, flank the desired gene sequence. The term therefore includes, for example, a recombinant DNA which is incorporated into a vector, into an autonomously replicating plasmid or virus, or into the genomic DNA of a prokaryote or eukaryote at a site other than its natural site; or which exists as a separate molecule (e.g., a cDNA or a genomic or cDNA fragment produced by PCR or restriction endonuclease digestion) independent of other sequences.

The peptides are prepared synthetically or by recombinant DNA technology. The term peptide is used interchangeably with polypeptide in the present specification to designate a series of amino acids connected one to the other by peptide bonds between the alpha-amino and alpha-carboxy groups of adjacent amino acids. Optionally, one or more peptide bonds are replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic yields a peptide mimetic, which is more stable and thus more useful as a therapeutic. Such mimetics, and methods of incorporating them into peptides, are well known in the art. Similarly, the replacement of an L-amino acid residue is a standard way of rendering the peptide less sensitive to proteolysis. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. The polypeptides or peptides are either in their neutral (uncharged) forms or in forms, which are salts, and either free of modifications such as glycosylation, side chain oxidation, or phosphorylation or containing these modifications, subject to the condition that the modification not destroy the immune stimulatory activity of the polypeptides.

Derivative peptide epitopes have an amino acid sequence, which differs from the amino acid sequence of a naturally occurring receptor peptide. Such derivative peptides have at least 50% identity compared to a reference sequence of amino acids, e.g., a naturally occurring glutamate receptor peptide. Preferably, a derivative is 90, 95, 98, or 99% identical to a naturally occurring protein sequence. The derivative contains a conservative amino acid substitution. By conservative substitutions is meant replacing an amino acid residue with another, which is biologically and/or chemically similar, e.g., one hydrophobic residue for another, or one polar residue for another. The substitutions include combinations such as Gly, Ala; Val, Ile, Leu; Asp, Glu; Asn, Gln; Ser, Thr; Lys, Arg; and Phe, Tyr. Nucleotide and amino acid comparisons described herein are carried out using the Lasergene software package (DNASTAR, Inc., Madison, Wis.). The MegAlign module used is the Clustal V method (Higgins et al., 1989, CABIOS 5(2):151-153). The parameter used is gap penalty 10, gap length penalty 10.

The invention provides significant advantages over standard methods of sciatic pain treatment. The methods described herein represent an effective, less invasive method of treatment without the potential for further nerve damage. Other advantages include fewer side effects compared to conventional therapeutic interventions. For example, epidural deposition of glutamate antagonists is associated with far fewer side effects than intravenous or subarachnoid infusions, as effects remain localized, as are the agonist effects of glutamate in the epidural space.

The methods are also applicable to pain related to degradation of cartilage in other joints, e.g., articulating joints such as a knee joint. For example, glutamate or glutamate receptor antagonists are administered directly into an articulating joint such as a knee or elbow to inhibit free glutamate from binding to glutamate receptors on neurons, thereby reducing pain in an individual suffering from or at risk of developing joint pain.

Other embodiments and features of the invention will be apparent from the following description thereof, and from the claims.

### DETAILED DESCRIPTION

Free glutamate is liberated from degenerating cartilage, a fibrous connective tissue derived from mesenchyme, which exists in several forms (hyaline cartilage, fibrocartilage, elastic cartilage). The free glutamate acts as a neurotransmitter. Glutamate binds to glutamate receptors on the surface of neurons and contributes to pain. Glutamate antagonists (administered epidurally or spinally) reduce pain such as sciatic pain resulting from herniated lumbar disc material in the spinal canal as well as other types of back pain. Human herniated disc material contains a significant concentration of extracellular glutamate.

The data described herein indicates that epidural glutamate infusion creates a localized hyperesthesia in an art-recognized animal (rat) model for human pain. The rat model is used to determine subtypes of glutamate receptors associated with changes in levels of nociception due to epidural glutamate. Glutamate antagonists are then evaluated to identify those, which effectively reduce signs of nociception in the animal model. Epidural and spinal injections of the glutamate antagonists are carried out and the level of sciatic or back pain evaluated.

Glutamate Receptors

Glutamate receptors are classified into categories based on the type of activation pathway triggered in the target neuron. Ionotropic receptors are receptor-channels, and the binding of glutamate of other specific agonists to the receptor protein opens up the pore-forming subunit of the receptor. Ionotropic receptors include NMDA receptors, AMPA receptors, and kainate receptors. Metabotropic receptors are receptors coupled with G proteins, and the binding of glutamate or specific agonists activates the G proteins and triggers or modulates one or another intracellular signalling pathway (InsP3/Ca²⁺ response or cAMP).

Ionotropic receptors are further classified based on the specificity of agonist binding. NMDA receptors are specifically activated by N-methyl-D-aspartate (NMDA), whereas non-NMDA receptors are not activated by this compound. The non-NMDA class of receptors include AMPA and KA receptors. AMPA receptors are activated specifically by α-amino-3-hydroxy-5-methyl-4-isoxalone propionate (AMPA), and KA receptors are activated specifically by kainate

The receptors include various subunits for each type or receptor. For example, for the AMPA receptor, there are 4 receptor subunits: GluR-1 to GluR-4 (also referred to as GluR-A to GluR-D). For KA receptors, there are the following subunits: GluR-5 to GluR-7 and KA-1 and KA-2. For NMDA receptors, there are 2 subunits: NR-1 and NR-2.

For metabotropic glutamate receptors (mGluRs) several types of receptors have been identified and cloned: mGluR1 and mGluR5 are positively coupled to the InsP3/Ca²⁺ pathway; and mGluR2, mGluR4, mGluR6 and mGluR7 are coupled negatively (i.e., inhibits) the adenylate cyclase (cAMP pathway) and/or VOCC activity. Metabotropic glutamate receptors in Group I include the following subtypes: mGlu1 and mGlu5. Those in Group II include mGlu2 and mGlu3; and those in Group III include mGlu4, mGlu5, mGlu7, and mGlu8. Antagonists bind to a heteromeric receptor complex or to one or more subunits or fragments thereof to inhibit signal transduction mediated the receptor, thereby leading to a reduction in perceived pain. For example, trans-1,2,-homo ACPD is a selective mGluR2 antagonist.

Dorsal root ganglion tissue has a rich concentration of glutamate receptors of at least three types of ionic receptors. By infusing glutamate subtype agonists (kainic acid, (α-amino-3-hydroxy, 5-methyl, 4-isoxazoleproprionate (AMPA), N-methyl-D-aspartate (NMDA), and metabotropic receptors, and measuring the extent of dorsal horn receptor expression by immunohistochemistry of glutamate receptors, and by performing von Frey fiber behavioral tests, a profile of receptor activity related to the presence of disc glutamate in the epidural space is obtained. Antagonists of both ionic and metabotropic receptors are available (NMDA receptors: MK-801; AMPA receptors: NBQX; kainate: LY382884 and ACEA-1011; and metabotropic receptors (L(+)-2-amino, 3-phosphonoproprionic acid (LAP-3), and (S)4-carboxy, 3-hydroxyphenyl glycine (CHPG)). These antagonists are infused with epidural glutamate to determine whether nociception is reversible by receptor antagonism.

Sciatic Pain and Lumbar Disc Herniations

Sciatic pain from lumbar disc herniations can be unbearable to patients even when the degree of mass effect on the nerve seems less than that seen in conditions of bony compression, as in lumbar spinal stenosis. In awake patients undergoing lumbar disc surgery, pressure on the root is not perceived as painful. Pressure on a nerve may create ischemia ^{and} breakdown of the basement membrane structure of the perineurium and dorsal root ganglion. This breakdown of basement membrane allows small molecules not otherwise found there to penetrate nerve cell membranes.

Cartilage Degradation in Disc and Other Joints

Disc cartilage, and cartilage in general, is unique in one particular way. It is the only tissue in the body that contains a matrix of carbohydrate and protein moieties in large extracelluar reservoirs unconstrained by cell membranes and intracellular metabolism. The molecular structure of this extracellular matrix has been elucidated. The hydrophilic qualities of healthy cartilage are related to the presence of aggrecan, i.e., the link and core proteins that are part of the larger proteoglycan matrix. Sequencing studies of these proteins show a composition of 30-50% glutamate and aspartate within the amino acid chain. The carboxyl moieties found in glutamate and aspartate maintain the hydrophilic qualities of these proteins. There are many metalloproteinases constituent in the epidural space that can enzymatically cleave these proteins, and disc degeneration is highly correlated with the loss of aggrecan.

Given the presence of high levels of glutamate within amino acid chains in disc material, and the presence of enzymatic systems for their degradation in the epidural space, studies were carried out to determine whether herniated disc material is a significant source of free glutamate from enzymatic degradation of aggrecan. Many types of glutamate receptors have been shown to have a role in sensory and pain transmission in primary afferent neurons. Free glutamate was found to be a “chemical” stimulus involved in lumbar radiculopathy by activating glutamate receptors located in the dorsal root ganglion and other regions of the spine in close proximity to degenerating cartilage.

Enzymatically-degraded glutamate is an important component of the sciatic pain process via effects on the dorsal root ganglion. Mechanical pain is also related to disc glutamate, e.g., by stimulating glutamate receptors found in the disc annulus or facets.

Free Glutamate in Human Disc Tissue

Studies were carried out determine whether free glutamate was present in surgical human disc specimens in significant concentrations. This was accomplished in two ways. First, immunofluorescent staining was performed with an anti-glutamate antibody. Disc material was defined as containing glutamate if regions of interest containing primary and secondary antibody demonstrated more immunofluorescence than sections with only primary antibody from the same disc specimen. Regions of interest were defined as larger than 10,000 pixels and free of cartilage cells. By this method, herniated disc specimens demonstrated specific glutamate immunostaining in disc matrix but no specific immunostaining for substance P.

Secondly, high performance liquid chromatography was performed on human disc specimens. Based on the wet weight of the specimens, average glutamate concentrations for free fragment discs were 0.18 mM and 0.11 mM for non-herniated central nuclear material. Free fragments from herniated discs had significantly higher concentrations than central nucleus preparations (P<0.001; by student's t-test).

These concentrations are biologically significant, since only during prolonged seizure activity are there similar concentrations of extracellular glutamate found in brain. To determine if baseline concentrations of glutamate in the extracellular space were normally higher or lower than this, and to determine whether the DRC was permeable to glutamate, the following rat model was used in further experiments. Anesthetized male Sprague-Dawley rats had miniosmotic pumps placed in the lower thoracic region with a P10 catheter tip in the lateral gutter of the epidural space. Radiolabeled glutamate at concentrations of 0.0003, 0.003, 0.03 and 0.22 mM was infused over a 72-hour period following implantation. Rats were euthanized by pentobarbital, followed by cardiac perfusion with 4% glutaraldehyde, and DRG were harvested with an operating microscope at the level of the catheter tip, and one level above and below bilaterally. Autoradiography of the six DRGs was performed in one animal with a 0.3 mM infusion.

Results confirmed that baseline epidural concentrations are much lower than concentrations of glutamate found in herniated disc material, since significant radiolabeling of the dorsal root ganglion occurred at concentrations as low as 0.003 mmol/L. At infusions below 0.22 mmol/L, significant radiolabeling occurred only on the side ipsilateral to the infusion catheter tip, indicating that such a mechanism leads to local nerve activation, e.g., as seen in clinical sciatica.

Further experiments were carried out to determine whether epidural glutamate is the cause of a hyperesthetic or nociceptive state. Using the rat epidural glutamate infusion model, both immunohistochemical and behavior tests were used to determine behavioral manifestations of a nociceptive state.

Immunohistochemical studies show expression of dorsal horn glutamate receptors in painful conditions involving the lower extremity in the rat. A relatively high concentration (2 mmol/L) of glutamate was infused for 72 hours (same infusion time period as in previous experiments) and densitometry was performed at 40× AMPA, NMDA and kainate for receptor expression at dorsal horn laminae I-III bilaterally, at spinal cord levels where the dorsal root ganglion input would enter the spinal cord dorsal horn, to determine whether receptor expression was increased. The microscopist was blinded to the nature of the sample. Using two-tailed T tests, these experiments showed an upregulation of expression over saline-infused controls for AMPA, NMDA and kainate receptors. When comparing ipsilateral to contralateral receptor expression by two-tailed t test, upregulation of receptor expression ipsilateral to the side of infusion is seen for kainate (p<0.05), AMPA (p<0.01), and NMDA (p<0.01) receptors, indicative of nociception.

Behavioral experiments have been completed at a wider range of concentrations. Rats are infused with epludial glutamate at concentrations of 2.0, 0.2, 0.02, 0.002 and 0.0002 mM for 72 hours (3 days). Von Frey fiber examinations were performed on left and right hind paws 24 hours before infusion and then 24, 72, and 144 hours after onset of glutamate infusion. The experimenter was blinded as to which infusate was used. Contralateral to ipsilateral differences were analyzed with respect to concentration of glutamate infusion and hours post-procedure. This analysis showed a significant hypersensitivity postoperatively, most prominent on day 3 but also present to a significant but lesser degree on postoperative day 1. The response was most significant at the 0.02 mM concentration but present at 0.002 and 0.2 mM concentrations. Significant differences in ipsilateral to contralateral responses in animals receiving the 0.02 mM/L glutamate infusion were seen on all postoperative days but were most prominent on day 3 after 72 hours of infusion (p<0.036; student's t test). Other glutamate concentrations showed less significant differences by this statistical method. Both statistical methods demonstrate a dose response curve with maximum nociceptive effects of glutamate at 0.02 mM/L.

The data indicate that free glutamate is present in herniated disc material and that this glutamate acts to potentiate pain by its effects at the dorsal root ganglion or other nearby regions of the back where glutamate receptors exist. Herniated disc material is a significant and enriched source of free glutamate, e.g., as a result of enzymatic action of metalloproteinases. Sources of epidural glutamate can significantly penetrate the dorsal root ganglion specifically on the ipsilateral side adjacent to the glutamate source. Elevated free glutamate concentrations surrounding nerve tissue creates physiological and behavioral change consistent with a hyperesthetic state in the distribution of the nerve.

Immunohistochemical and Densitometry Studies

Glutamate is infused at concentrations of 02, 0.02, and 0.002 mM at 72 hours after implantation and immunohistochemical and densitometry studies carried out to determine if there is a concentration-related change in receptor expression that could correlate with concentration dependencies seen in behavioral studies. Densitometry analyses are carried out in blinded fashion on five sections per animal (n 5) for a total of 25 observations per side at each concentration. Behavioral studies are then be performed focusing on the use of receptor agonists AMPA, NMDA, and kainic acid in infusion concentrations ranging from 2.0 mM to 0.002 mM using methods known in the art, e.g., the methods described by Hu et al., 1998, Pain 77:15-23. In some experiments, an additional condition, placing a spacer in the neural foramen at the level and ipsilateral to the catheter tip, is included.

Tissue sections of spinal cord at 72 hours post-infusion are analyzed for glutamate receptor expression in dorsal horn laminae I-III, to determine if they correlate well with behavioral data by microscopists blinded to experimental exposures.

Depending on which of the ionotropic receptor agonists manifest behavioral or physiological signs of a local ipsilateral hyperesthetic state, behavioral and immunohistochemical tests are repeated using glutamate infusion with specific glutamate receptor antagonists, including metabotropic glutamate antagonists (possibilities include MK-801 for NMDA antagonism; GYK152466, CNQX or NBQX for AMPA antagonism; ACEA-1011, LY294486, or LY382884 for kainic acid; CHPG and MPEP for metabotropic receptors antagonism). Experiments use concentrations of antagonists that are 4× glutamate concentration to assure adequate receptor blockade. In addition to von Frey tests, animals are tested for their ability to navigate a maze pre-operatively and at 72 h post-infusion to determine if there are signs of generalized central nervous system toxicity. Immunohistochemical analysis of these animals is carried out to evaluate receptor expression 72 hours post-infusion.

To evaluate human responses to glutamate antagonist treatment, subjects are tested by a visual analog pain scale and the SF-36 health questionnaire 24 hours prior to injection, and then at 4 hours, 24 hours and 7 days after injection of either antagonist or placebo. Injection is performed via a transforaminal approach at the 6 o'clock position within the pedicle as seen on AP fluoroscopy.

Specific methods using an art-recognized animal model for pain are carried out as follows.

Implantation of an Epidural Alzet Miniosmotic Trump for Epidural Infusion and Placement of Foraminal Stents

Female Sprague-Dawley rats, 300 to 500 grams, are epidurally and unilaterally infused with glutamate in the L5/S1 level for 72 hours via a subcutaneously implanted Alzet miniosmotic pump in concentrations of 0.002, 0.02, 0.2, or 2 mM. This range is chosen because human herniated disc material has an average glutamate concentration of 0.18 mM, and baseline concentrations of glutamate in the epidural space are lower than micromolar concentrations.

Induction of anesthesia is by 4% Halothane and maintenance by 1.5% Halothane. When a surgical level is obtained, the animal is placed prone and the back is shaved and washed with Betadine. Following sterile procedure, a midline incision 2 cm in length is cut through the skin with scalpel and scissors. The paraspinous muscles are retracted locally and a small laminectomy is made on one side of the lamina at T10 exposing the dura and nerve roots. A P50 catheter fused proximally to a P10 catheter, which in turn is secured to an Alzet miniosmotic pump, is placed in the epidural space on that side. A 4.0 nylon suture is looped around the catheter and stitched to paraspinous muscle to prevent dislodgement of the catheter from the epidural space. Any slack tubing is loosely coiled and secured with sutures to the paraspinous fascia. A small pocket posterior to the laminectomy is made subcutaneously with scissors for the miniosmotic pump. The pump itself is secured in place to the fascia. The pump is sterile and filled with 100 μL of one of the following: Normal saline (control); one of three different concentrations (0.02, 0.20, or 2.00 mM) of glutamate dissolved in saline, one of three different concentrations of an antagonist to glutamate, (either ionotropic or metabotropic) dissolved in saline. A series of experiments is run with glutamate and antagonists added together and dissolved in saline. The flow rate of infused compounds is 1 μL/hour for 72 hours. The skin and subcutaneous tissue are closed as a single layer in interrupted fashion with 3.0 nylon Atipamezole (1 mg/kg) is given I.P. at the end of the procedure. The animal is kept warm and continuously observed in the Neurosurgery operative suite until fully alert and ambulatory. The animal is then placed in the Central Research facility where food and water access is assured, and buprinorphine (0.03-0.05 mg/kg) is administered IM to relieve any signs of incisional discomfort. The rat is killed immediately by pentobarbital injection (150 mg/kg into the peritoneum) if signs of paralysis or other stresses such as biting or scratching at the wound site are seen. Behavioral studies are performed until euthanization 72 hours after surgery.

A series of rats have a stainless steel rod inserted at the intervertebral foramen next to the L5 DRG. The rod compresses the neurons innervating the plantar surface of the hind leg muscles and provide an additional mode to study mechanical hyperalgesia.

Von -Frey Fiber Testing

Behavioral Tests—The von Frey Fiber mechanical allodynia assay is performed 24 hours preoperatively and 24, 72, and 144 hours postoperatively. The plantar surface of each paw is tested for pain response. The von Frey fiber test kit has plastic fibers of different widths, each conveying different amounts of force. In total, ten of the fibers are utilized in this experiment. Starting with 0.6 grams of force and working up to 1, 1.4, 2, 4, 6, 8, 10, 15, and finally 26, each paw's response is recorded. Paw withdrawal movement at lower applied force is considered a hyperalgesic response to prodding with the von Frey Fiber.

The protocol has the experimenter tap the bottom surface of the paw with one fiber at a time for six seconds each. The rats are housed in elevated metal cages with grids on the bottom so that the initial fiber tested is that eliciting 0.6 grams of force. If a response is not recognized, then the next fiber (one that elicited 1.0 grams of force) is applied, and so on in increasing order of force until a paw withdrawal response was recorded. After the initial response is recorded, the experimenter completes the testing procedure by testing the same paw with fibers in descending order of force. This is done until no response is elicited. The final result is the lowest amount of force needed to produce a withdrawal response.

Starting with the left hind paw, this protocol is repeated for the right hind paw, the left front paw, and the right front paw. Again, an inverse relationship between force and paw withdrawal is hypothesized. With increased amounts of glutamate injected, the force necessary to produce a response is hypothesized to decrease, indicative of a greater sensitivity to pain with the presence of increased amounts of glutamate.

The pre-operation test 24 hours prior to the insertion of the pump is used as a control measurement. The rat's weight is recorded as a baseline, to allow the experimenter to detect any drastic changes. If the rat's weight decreases by over 50 grams, the rat is considered ill and its data discarded. After the initial weighing, the rats are placed into the metal cages where the Von Frey fiber assay will be conducted. This placement, usually for half an hour, is for adaptation purposes. Without adaptation to the strange, new environment of the cages, the rats wander around the cages making it difficult to record any accurate Von Frey fiber results.

Harvest of Spinal Cord and Dorsal Root Ganglion

For euthanization, the animal is anesthetized with pentobarbital 150 mg/kg. A supradiaphragmatic incision is made in the rib cage exposing the heart within the mediastinum. The right ventricle is pierced with a 16 gauge perfusion needle and is secured with a clamp as a buffered 4% paraformaldehyde solution is infused with a perfusion pump for at least 2 minutes and until the tissues have hardened sufficiently.

Tissues are harvested by enlarging the laminectomy with the carcass prone. The site of the catheter tip is noted with relation to the spinal cord and closest ipsilateral dorsal root ganglion. Under microscopic magnification, the spinal cord is cut away from surrounding nerve roots and is lifted in a single piece. The most proximal region is at the level of the next proximal dorsal root ganglion and the distal end at the level of the dorsal root ganglion below. The spinal cord is nicked with a knife at the proximal end and a silt is made over the left ventral horn for orientation identification. Dorsal root ganglia are separately harvested, as are the brains.

Immunohistochemistry and Densitometry Determinations

Dependent upon tissue preparation requirements, animals are sacrificed by two methods. For analyses that require Immediate fixation, spinal cord tissue will be fixed by cardiac perfusion of a tissue fixative solution. The cardiac perfusion, following pentobarbital overdosing, consists of the administration of a 200 ml bolus of heparinized saline into the left ventricle of the heart followed by the perfusion of 300 ml of 10% neutral buffered formalin or 4% paraformaldehyde solution. When spinal cord tissue is collected for NDA and protein analyses, the procedure is similar except that the deeply anesthetized rat is decapitated. The spinal cord is then briefly immersed in liquid nitrogen. After thawing over a 3 minute period, the cord is transected and separated from nerve roots and epidural fat and veins. The tissue is placed in a −70° C. methylbutane bath for 30 seconds, wrapped in parafilm and foil, and stored in liquid nitrogen.

Therapeutic Administration of Glutamate Receptor Antagonist Compounds

Glutamate receptor antagonist compounds described herein are useful to inhibit binding of free glutamate from cartilage degradation in disc or joint tissue from binding to glutamate receptors on nerve cells. When a peptide is used as an antagonist, it is administered to a patient in the form of a peptide solution in a pharmaceutically acceptable carrier. Such methods are well known to those of ordinary skill in the art. The peptides are administered at an intravenous dosage of approximately 1 to 100 μmoles of the polypeptide per kg of body weight per day. The compositions of the invention are useful for parenteral administration, such as intravenous, subcutaneous, intramuscular, intraperitoneal, or directly into a joint or area surrounding a herniated disc. Preferably, the antagonists are administered epidurally, spinally, or directly into a joint space (e.g., a knee joint space or an elbow joint space). A pain-relieving dose of the peptide ranges from 0.1 to 100 mg, which may be administered at one time or repeatedly to a patient. A plurality of peptides are optionally administered together (simultaneously or sequentially).

Peptides are recombinantly produced or synthetically made using known methods. Peptide solutions are optionally lyophilized or granulated with a vehicle such as sugar. When the compositions are administered by injection, they are dissolved in distilled water or another pharmaceutically acceptable excipient prior to the injection.

DNA encoding a peptide antagonist may also be administered, e.g., by incorporating the DNA into a viral vector. Nucleic acids are administered using known methods, e.g., intravenously, at a dose of approximately 10⁶ to 10²² copies of the nucleic acid molecule.

Preferably, the antagonists are relatively small organic compounds, e.g., ±)-trans-1-Amino-1-carboxycyclopentane-2-acetic acid (trans-1,2-homo-ACPD; M.W. 187.17), a highly selective mGluR2 antagonist; L(+)-2-Amino-3-phosphonopropionic acid (L-AP3; M.W. 169.07), a selective antagonist of the phosphoinositide-linked metabotropic glutamate response; AMPA-KA antagonist LY293558, a group II metabotropic glutamate receptor selective agonist; or YM872 ([2,3-dioxo-7-(1H-imidazol-1-yl)-6-nitro-1,2,3,4-tetrahydroquinoxalin-1-yl]acetic acid monohydrate, a competitive AMPA receptor antagonist. Dosage determination and excipient choice is well within the skill of those practicing in the art of medicine and pharmaceuticals.

The pain-relieving composition preferably contains a receptor antagonist specific for one glutamate receptor subtype and does not contain a receptor antagonist specific for other subtypes. Alternatively, the composition contains a mixture of antagonists with specificity for two or more different glutamate receptor subtypes.

Other embodiments are within the following claims.

What is claimed is:

1. A method of alleviating pain in a mammal, comprising contacting a neuronal cell of a cartilaginous tissue with an antagonist of a glutamate receptor, wherein inhibition of binding of free glutamate to said receptor on said neuronal cell alleviates pain.

2. The method of claim 1, wherein said glutamate receptor is an ionotropic glutamate receptor.

3. The method of claim 2, wherein said ionotropic glutamate receptor antagonist is a non-N-methyl-D-aspartate (NMDA) type receptor antagonist.

4. The method of claim 2, wherein said non-NMDA receptor antagonist is chosen from the group consisting of a (S)-a-amino-3-hydroxy-5-methyl-4-isoxalone propionate (AMPA) receptor antagonist and a kainate-activated (KA) receptor antagonist.

5. The method of claim 1, wherein said antagonist is an NMDA receptor antagonist.

6. The method of claim 5, wherein said NMDA receptor antagonist is MK-801.

7. The method of claim 4, wherein said AMPA receptor antagonist is selected from the group consisting of GYK152466, CNQX, and NBQX.

8. The method of claim 4, wherein said KA receptor antagonist is selected from the group consisting of LY294486, LY382884 and ACEA-1011.

9. The method of claim 1, wherein said glutamate receptor is metabotropic glutamate receptor.

10. The method of claim 1, wherein said antagonist is a metabotropic glutamate receptor antagonist selected from the group consisting of L(+)-2-amino, 3-phosphonoproprionic acid (LAP-3) and (S)4-carboxy, 3-hydroxyphenyl glycine (CHPG).

11. The method of claim 1, wherein said antagonist preferentially inhibits binding of free glutamate to a mGlu2 receptor.

12. The method of claim 1, wherein said pain is selected from the group consisting of back pain, joint pain, and sciatic pain.

13. The method of claim 1, wherein said neuronal cell is a dorsal root ganglion cell.

14. The method of claim 1, wherein said cartilaginous tissue is intervertebral disc tissue.

15. The method of claim 1, wherein said cartilaginous tissue is articulating joint tissue.

16. The method of claim 1, wherein said articulating joint tissue is knee joint tissue.

17. The method of claim 1, wherein said articulating joint tissue is elbow joint tissue.

18. The method of claim 1, wherein said glutamate antagonist is administered directly into an epidural space.

19. The method of claim 1, wherein said glutamate antagonist is administered into spinal fluid.

20. The method of claim 1, wherein said glutamate antagonist is administered into a joint space of an articulating joint.
